(19) United States
(12) Patent Application Publication
(10) Pub. No.: US 2009/0156916 A1
(43) Pub. Date: Jun. 18, 2009

(54) CATHETER SYSTEMS WITH BLOOD MEASUREMENT DEVICE AND METHODS

(76) Inventors: Huisun Wang, Maple Grove, MN (US); Todd Stangenes, Minneapolis, MN (US)

Correspondence Address: SJM/AFD - TRENNER, 14901 DEVEAU PLACE, MINNETONKA, MN, 55345-2126, US

(21) Appl. No.: 12/345,590

(22) Filed: Dec. 29, 2008

Related U.S. Application Data
(63) Continuation-in-part of application No. 11/959,214, filed on Dec. 18, 2007.

(51) Int. Cl. A61B 5/1459; A61B 5/15

(52) U.S. Cl. 600/339; 600/364; 600/341

(57) ABSTRACT

Catheter systems with blood measurement device and methods are disclosed. An exemplary catheter system for use in positioning a distal end of a catheter body at a desired location in a patient's body may include a needle provided at the distal end of the catheter body to withdraw blood from the patient's body. The needle is fluidically connected to a proximal end of the catheter body. The catheter system may also include a measurement device provided at the proximal end of the catheter body. The measurement device is configured to receive blood withdrawn by the needle for measuring a blood gas value of the blood for use in positioning the distal end of the catheter body at the desired location in the patient's body.

### CROSS-REFERENCE TO RELATED APPLICATIONS

This application is a continuation-in-part of U.S. application Ser. No. 11/959,214, filed 18 Dec. 2007, now pending (the '214 application). The '214 application is hereby incorporated by reference in its entirety as though fully set forth herein.

### BACKGROUND OF THE INVENTION

a. Field of the Invention

The present invention relates generally to catheter positioning and, in particular, to determining or verifying that a catheter is positioned at a desired location or atrium of the heart, based on measurements (e.g., parameters such as oxygen saturation, carbon dioxide concentration, pH, or the like) of the blood or perfused tissue. The invention is also useful for in vivo blood measurements independent of any guidance objective.

b. Background Art

Catheters have been in use for medical procedures for many years. Catheters can be used for medical procedures to examine, diagnose, and treat while positioned at a specific location within the body that is otherwise inaccessible without more invasive procedures. During these procedures a catheter is typically inserted into a vessel near the surface of the body and is guided to a specific location within the body for examination, diagnosis, and treatment. For example, catheters can be used to convey an electrical stimulus to a selected location within the human body, e.g., for tissue ablation. Catheters with sensing electrodes can be used to monitor various forms of electrical activity in the human body, e.g., for electrical mapping.

Catheters are used increasingly for medical procedures involving the human heart. Typically, the catheter is inserted in an artery or vein in the leg, neck, or arm of the patient and threaded, sometimes with the aid of a guide wire or introducer, through the vessels until a distal tip of the catheter reaches the desired location for the medical procedure in the heart. In the normal heart, contraction and relaxation of the heart muscle (myocardium) takes place in an organized fashion as electro-chemical signals pass sequentially through the myocardium.

Sometimes abnormal rhythms occur in the heart, which are referred to generally as arrhythmia. The cause of such arrhythmia is generally believed to be the existence of an anomalous conduction pathway or pathways that bypass the normal conduction system. These pathways can be located in the fibrous tissue that connects the atrium and the ventricle.

An increasingly common medical procedure for the treatment of certain types of cardiac arrhythmia is catheter ablation. During conventional catheter ablation procedures, an energy source is placed in contact with cardiac tissue (e.g., associated with an anomalous conduction pathway) to create a permanent scar or lesion that is electrically inactive or noncontractile. The lesion partially or completely blocks the stray electrical signals to lessen or eliminate arrhythmia.

Ablation of a specific location within the heart requires the precise placement of the ablation catheter within the heart. Precise positioning of the ablation catheter is especially difficult because of the physiology of the heart, particularly because the heart continues to beat throughout the ablation procedures. Commonly, the placement of the catheter is guided by fluoroscopy sometimes using a contrast agent and/or by a combination of electrophysiological guidance and computer generated maps/models that may be generated during a mapping procedure. Additionally, in some cases, ultrasonic guidance is provided by introducing an ultrasound transducer to the procedure site via a separate catheter. Even with these guidance techniques, proper positioning of the distal end of the catheter for certain procedures may still involve considerable uncertainty. Moreover, these guidance techniques may complicate the procedure or expose the patient to increased risk, additional procedures or inconvenience.

### BRIEF SUMMARY OF THE INVENTION

The present invention recognizes that positioning of the distal end of a catheter at a desired location can be determined or verified through measurement of blood gas values proximate to the distal end. In particular, it is expected that venous blood will have a lower oxygen saturation (and higher carbon dioxide concentration) than arterial blood. Accordingly, blood gas measurements or changes therein may be useful to indicate that the distal end of the catheter is positioned in a vein or the right side of the heart (e.g., the right atrium), on the one hand, or in an artery or the left side of the heart (e.g., in the left atrium), on the other. This information may be useful in certain catheter guidance applications.

The case of the transeptal procedures is illustrative. Access to the left atrium and pulmonary veins often requires performing a transeptal procedure where a catheter or other instrument is pushed through the interatrial septum between the left and right atriums. Such an instrument preferably punctures the septum at its thinnest location, for example, the fossa ovalis. This location is not readily determined using conventional imaging techniques such as fluoroscopy or intracardial mapping. Instead, the physician determines the puncture location based on his/her experience using the catheter to probe the interatrial septum to identify the most compliant location, typically the fossa ovalis. Such experience only comes with time, and may be quickly lost if the physician does not perform the procedure on a regular basis.

It will thus be appreciated that confirmation that the interatrial septum has been penetrated and that the distal end of the catheter is in the desired atrium may be useful to a physician. In this example, passage of the distal end of the catheter from one atrium to the other by traversing the interatrial septum will generally be accompanied by a transition from contact with deoxygenated venous blood to well oxygenated arterial blood or vice-versa. Accordingly a blood gas measurement, e.g., an in vivo measurement, or a monitored change in an associated value, can indicate that the distal end of the catheter is positioned in the correct atrium for the procedure under consideration.

In addition, such in vivo measurements may be useful in monitoring a patient independent of catheter guidance functionality. Indeed, such in vivo measurements may be more reliable than conventional pulse oximetry measurements which attempt to distinguish effects due to arterial blood from effects associated with other absorbers/attenuators, and that can be difficult in cases of patient motion and low perfusion.

Blood gas measurements can be made, for example, using optical or chemical processes, and any appropriate measurement can be employed in the context of the present invention. By way of example, oxygen saturation can be measured optically. In particular, oxygenated blood has different light transmission or absorption characteristics than deoxygenated blood. This is reflected in the observation that well-oxygenated arterial blood appears bright and red whereas deoxygenated or venous blood appears dark and bluish. Optical techniques that provide an indication of color or color change may therefore be used to measure oxygen saturation in vivo, to determine catheter position and/or to guide a catheter as discussed above.

Conventional oximeters typically utilize optical sources (e.g., LEDs) of two or more wavelengths. The sources are used to illuminate perfused tissue. The resulting optical signals are detected after they have been transmitted through or reflected from the perfused tissue. In either case, the optical signals are attenuated due to interaction with the patient's blood/perfused tissue. In these applications, the ratio of an attenuation related value for the red signal to a similar value for the infrared signal can be used to compute oxygen saturation.

It may be expedient to use conventional oximetry processing in this regard and the resulting values are useful for patient monitoring. However, simplified processes may be adequate for the noted objective of catheter positioning. In particular, it is expected that oxygen saturation in the left atrium will be very high, generally above 95% and often at least about 99%. On the other hand, oxygen saturation in the left atrium will be considerably lower, generally below 90% and often below about 80%. Accordingly, high accuracy is not necessary to distinguish between the atria.

Moreover, an at least partially catheter-borne instrument can directly access the patient's blood substantially without interference associated with other optical attenuators. Accordingly, various processing associated with addressing variations in optical signal wavelengths, certain conventional pulse oximetry signal-to-noise ratio, addressing patient motion and the like may be unnecessary. Indeed, the conventional use of multiple optical sources at specific red and infrared wavelengths may be unnecessary. However, as noted above, the use of conventional instrumentation and processing may be expedient and provides information useful for patient monitoring.

Thus, in accordance with one aspect of the present invention, a method is provided for use in positioning a catheter at a desired location in a patient's body. The method may comprise inserting the catheter into the patient's body. The method may also comprise withdrawing blood from the patient's body at a distal end of the catheter, and transferring the withdrawn blood through the catheter to a proximal end of the catheter. The method may also comprise measuring at least one parameter in the withdrawn blood at the proximal end of the catheter while the catheter remains inserted in the patient's body. The at least one measured parameter may be used to assist in positioning the catheter at the desired location in the patient's body.

In accordance with another aspect of the present invention, a catheter system for use in positioning a distal end of a catheter body at a desired location in a patient's body comprises a needle provided at the distal end of the catheter body to withdraw blood from the patient's body. The needle is fluidically connected to a proximal end of the catheter body. A measurement device is provided at the proximal end of the catheter body. The measurement device is configured to receive blood withdrawn by the needle for measuring a blood gas value of the blood for use in positioning the distal end of the catheter body at the desired location in the patient's body.

In accordance with yet another aspect of the present invention, a catheter system may include a catheter body having a proximal end and a distal end, the proximal end having a handle for positioning the proximal end in a patient's body. A needle is insertable through the catheter body to the distal end of the catheter body, the needle configured to withdraw blood from the patient's body near the distal end of the catheter body. A measurement device is provided at the proximal end of the catheter body, the measurement device measuring a blood gas value of the blood. An output device is operatively associated with the measurement device. The output device is configured to output information based on the blood gas value for use in positioning the distal end of the catheter body at the desired location in the patient's body.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic view of a catheter system incorporating an oximeter in accordance with one embodiment of the present invention.

FIG. 2 is a side cross-sectional view of a catheter handle of the catheter system incorporating the oximeter in another configuration.

FIG. 3 is a side cross-sectional view of a sensor/detector for an oximeter which may be used with the catheter handle shown in the configuration of FIG. 1 or FIG. 2.

FIG. 4 is a side view of another catheter handle which may be used in accordance with another embodiment the present invention.

FIG. 5 is a side cross-sectional view of a sensor/detector for an oximeter which may be used with the catheter handle shown in FIG. 4.

FIG. 6 is a side cross-sectional view of another sensor/detector for an oximeter which may be used with the catheter handle shown in FIG. 4.

FIG. 7 is a flow chart illustrating a process for performing a medical procedure using a catheter with oximeter in accordance with the present invention.

### DETAILED DESCRIPTION OF THE INVENTION

The present invention relates to certain structure and methodology for using blood gas measurements to assist in positioning a catheter for a medical procedure. A variety of blood gas measurements may be performed in this regard, including oxygen saturation measurements, carbon dioxide concentration measurements or other blood gas measurements, and these measurements may be performed optically, chemically or in any other appropriate manner. In addition, a variety of types of medical procedures may be assisted in this regard including, for example, diagnostic and therapeutic procedures. In the following description, the invention is set forth in the context of a catheter including oximetry structure for obtaining oxygen saturation measurements. It is noted that the term “catheter” is used broadly herein to include various types of catheters (e.g., any of a wide variety of ablation catheters) and also includes the various components that make up a typical catheter (e.g., the guiding introducer, electrode, etc.). Moreover, the invention is described with respect to specific procedures including transseptal procedures. While this structure and these applications represent an advantageous context for application of the present invention, it will be appreciated that the invention is not limited to such structure and applications. Accordingly, the following description should be understood as providing an exemplary discussion of the invention and not by way of limitation.

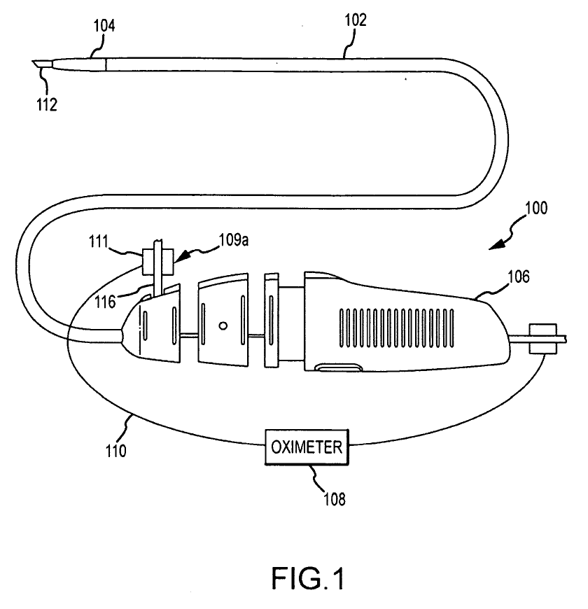

FIG. 1 illustrates a catheter system 100 in accordance with the present invention. The catheter system 100 includes a catheter 102 having a distal end 104 for introduction into a patient to a desired location, and a handle 106 at the proximal end. As used herein, the term “distal” refers to a portion inserted adjacent the target tissue within the body of a patient and away from the physician or other user (referred to generally as a “clinician”). Also as used herein, the term “proximal” refers to a portion directed toward the clinician (and opposite or away from the portion inserted within the body of a patient).

The catheter 100 may be introduced and guided into the patient through an artery or vein, for example, in the patient's neck, arm or leg, and then threaded through the vessel to the patient's heart. As discussed above, for transseptal procedures, the distal end 104 of the catheter 102 penetrates the interatrial septum to gain access to the desired location for a medical procedure such as an ablation procedure to correct cardiac arrhythmia.

One application of the present invention is to provide an indication to a physician that the distal end 104 of the catheter 102 is positioned either in the left atrium or the right atrium. This is accomplished by obtaining blood gas measurements that are readily used to distinguish between the deoxygenated venous blood of the right side of the heart, including the right atrium, from the well-oxygenated arterial blood of the left side of the heart, including the left atrium.

In the embodiments described below, optical oximetry measurements are used in this regard. Such measurements may measure oxygen saturation or carbon dioxide concentration. Moreover, these measurements may be simple color or attenuation measurements or may be pulsatile waveform or photoplethysmographic measurements. In the implementations described below, an oximeter is used to make conventional photoplethysmographic measurements so as to determine oxygen saturation.

The oxygen saturation of arterial blood, or SₐO₂, is readily distinguished from the oxygen saturation of venous blood, SᵥO₂, particularly where such measurements are performed in the left and right atria. In particular, for healthy patients, it is expected that the measured value of SₐO₂ will generally be in excess of 95% and often about 99% or more. By contrast, the measured value of SᵥO₂ is expected to be below 90% and often below about 80%. Accordingly, any appropriate observations can be used to indicate the position of the distal end of the catheter or the transition between the atria including threshold comparisons or changes in measured oxygen saturation.

Thus, for example, a physician may monitor oxygen saturation readings during a transseptal procedure to identify a change in oxygen saturation indicating a transition from arterial blood to venous blood or vice versa. For example, a change in measured oxygen saturation of at least 5% and, more preferably, at least 10% may indicate a transition between the atria. Additionally or alternatively, a physician may use an oxygen saturation measurement to confirm the position of the distal end of the catheter that has been preliminarily determined by the physician based on an imaging system or tactile feedback indicating that the interatrial septum has been penetrated. For example, the physician may base this determination on a comparison to a threshold of, for example, 90% oxygen saturation or some other value including a patient-dependent value.

This oxygen saturation monitoring process may also be at least partially automated. In this regard, the oximeter may execute algorithms to identify specified conditions. For example, the physician or other person involved in the medical procedure may use a user interface to identify a procedure to be performed, e.g., a transseptal procedure, and request notification when the distal end of the catheter has reached the desired location. The physician may define thresholds to be utilized for making this determination or default thresholds may be defined in the processing logic. In either case, the logic may monitor oxygen saturation readings to identify an appropriate condition, e.g., transition of the oxygen saturation readings from below 90% (or other threshold) to above 90%, or a change in the monitored oxygen saturation value of at least 5% or at least 10%. Averaging filters may be used in this regard to distinguish between transient changes, for example, triggered by patient motion or other artifact, and persistent changes that more likely indicate passage of the distal end of the catheter across the interatrial septum. Whatever the condition is that is defined by the logic, when the condition is satisfied, an indication may be provided to the physician in any appropriate way. For example, an audio or visual output may be provided by the oximeter or a vibration device in the catheter handle may be triggered to provide a tactile indication to the physician.

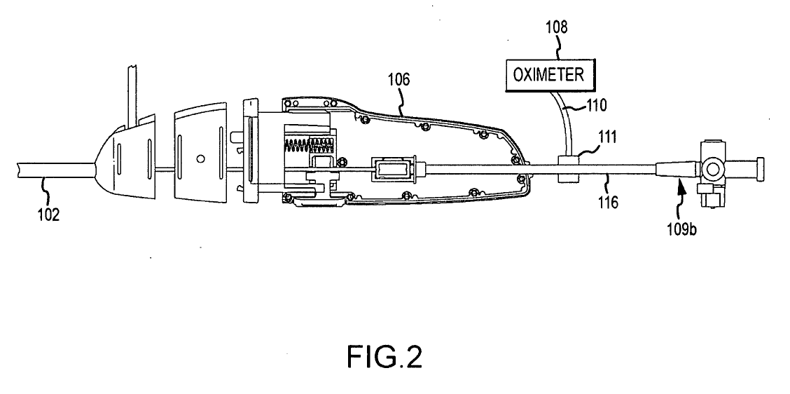

Accordingly, the illustrated catheter system 100 may also include an oximeter 108 which performs the oxygen saturation calculations and executes other logic, as noted herein. Referring again to FIG. 1, the oximeter 108 is shown in a first configuration wherein the oximeter is connected to a first port 109a on the catheter handle 106. FIG. 2 is a side cross-sectional view of a catheter handle of the catheter system incorporating the oximeter in another configuration. In FIG. 2, the oximeter 108 is connected to a second port 109b on the catheter handle 106. In either case, blood may be withdrawn at the distal end 104 of the catheter body 102 and delivered through the catheter body 102 to the port 109a or 109b in order to measure one or more parameter of the blood. In one embodiment, blood is delivered through the lumen of the catheter sheath to port 109a (e.g., during an ablation procedure), and blood is delivered from the needle 112 to port 109b (e.g., during the transceptal placement procedure).

In one embodiment, the oximeter 108 may be operatively associated with an optical detector. Such an optical detector detects optical signals transmitted through or reflected by the patient's blood. In either case, the optical signals are attenuated by the patient's blood in a manner which allows for calculation of oxygen saturation.

As is conventionally known, the optical signals utilized by the oximeter 108 may be transmitted by LEDs, for example, a red LED and an infrared LED. In such a case, a drive signal for driving the sources is located in the oximeter 108. An optical detector in the oximeter 108 receives the optical signals and generates an electrical output signal representative of the received signals. It will be appreciated that, depending on the implementation, analog or digital signals may be used in this regard. Additionally, in certain applications, wireless signals may alternatively be used in this regard. Depending on the implementation, the system 100 may also include an interface (not shown) for displaying or otherwise processing output from the oximeter 108.

The illustrated system 100 includes optical connections 110. The optical connections 110 may be optical fiber(s) coupled between the source/detector on one end, and the oximeter 108 on the other end. For example, each optical source may be optically coupled to a corresponding optical fiber using appropriate optical elements such as lenses or mirrors. Alternatively, multiple sources may be coupled to a single optical fiber by use of diffraction gradings, prisms, mirrors or the like, so as to provide a wavelength multiplexed signal. It will be appreciated that this signal may also be time division multiplexed, frequency division multiplexed or code division multiplexed. That is, each source is typically pulsed in a manner that allows for distinguishing between the contributions of each source to the detector signal and also for reducing noise.

The oximeter 108 is described above as it may be implemented as a reflectance oximeter. In a reflectance oximeter, optical signals are transmitted into the patient's blood and reflected back to the detector or detector fiber ends. Alternatively, a transmittance-based oximeter may be employed where the optical signals are transmitted through the patient's blood to a detector disposed opposite the sources or source fiber ends.

Before continuing, it is noted that the catheter system 100 may further include a tip electrode (not shown), such as an ablation electrode, and electric wiring for electrically coupling the tip electrode to an energy source. Although not shown, the electrical wiring would be threaded through the catheter body 102 to the handle 106. It should be noted that the ablation catheter may use radio frequency (RF), cryo, microwave, ultrasonic or laser technologies. The electrical wiring may convey electrical signals from the sensor(s) to a data acquisition/processing/output device (also not shown), such as, e.g., an echocardiogram (ECG) device. In addition, for certain applications, irrigation openings may be provided at the distal end 104 of the catheter 100 for irrigated procedures. In such cases, appropriate fluid channels are also provided through the catheter to the openings.

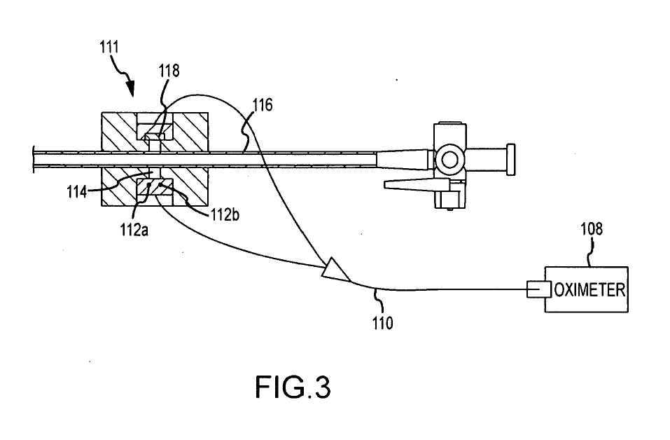

FIG. 3 is a side cross-sectional view of a sensor/detector 111 for an oximeter 108 which may be used with the catheter handle shown in the configuration of FIG. 1 or FIG. 2. That is, the oximeter 108 may be implemented at either ports 109a (shown in FIG. 1) or the port 109b (shown in FIG. 2). The sensor/detector 111 includes a red LED 112a, an infrared LED 112b and a photo detector 118.

In operation, drive signals transmitted via the electrical wiring 110 cause sources 112a, 112b to flash according to a defined multiplexing scheme. In this regard, the resulting optical signals may be time division multiplexed such that the sources 112a, 112b are alternately flashed, generally with a dark interval in between. Alternatively, pulse oximeters may be frequency division multiplexed or code division multiplexed.

In any event, the resulting optical signals are transmitted via a substantially transparent covering 114 into the patient's blood within tubing 116. A portion of these optical signals is received at the photo detector 118. The photo detector receives the incoming optical signals, generates an electrical signal representative of the received optical signals and transmits the electrical signal back to the oximeter. Optionally, some signal processing and conditioning may be performed at the photo detector 118. For example, the signal may be converted from a current signal to a voltage signal, amplified, digitized or the like, Alternatively such signal processing may be performed at the oximetry instrument. Additional functionality such as separating the received signal into AC and DC components, de-multiplexing the signal, filtering, removing motion or other artifact and executing algorithms for calculating a value related to oxygen saturation may be performed by a processing unit, generally located at the oximeter.

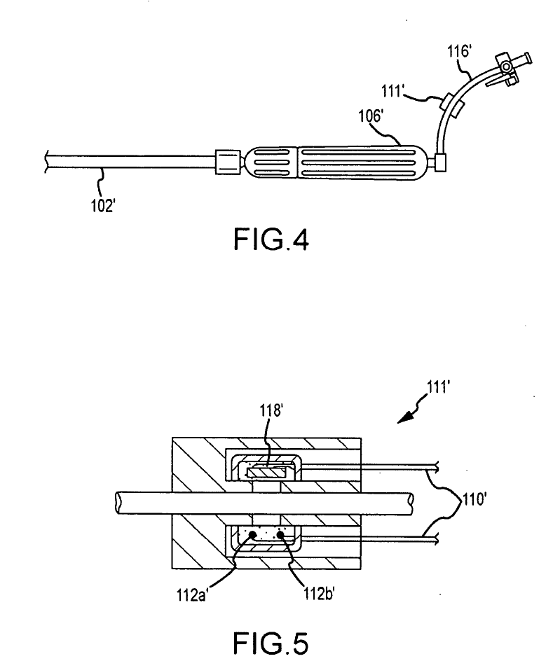

FIG. 4 is a side view of another catheter handle 106′ which may be used in accordance with another embodiment the present invention. The catheter handle 106′ may be the Agilis™ Steerable Introducer (commercially available from St. Jude Medical). FIG. 5 is a side cross-sectional view of a sensor/detector for an oximeter which may be used with the catheter handle shown in FIG. 4. The sensor/detector 111′ is located on an extension tube 116′ and can be used to detect the location of the distal portion of the catheter body 102′. In FIG. 4 and FIG. 5, previously described parts are designated with the “prime” indicator and are not described herein again.

These configurations of catheter systems in which the oximeter may be implemented are shown merely for purposes of illustration and are not intended to be limiting in any regard. Still other configurations are also contemplated as being within the scope of the invention described herein. Such configurations may depend on a variety of design characteristics, as will be readily appreciated by those having ordinary skill in the art after becoming familiar with the teachings herein.

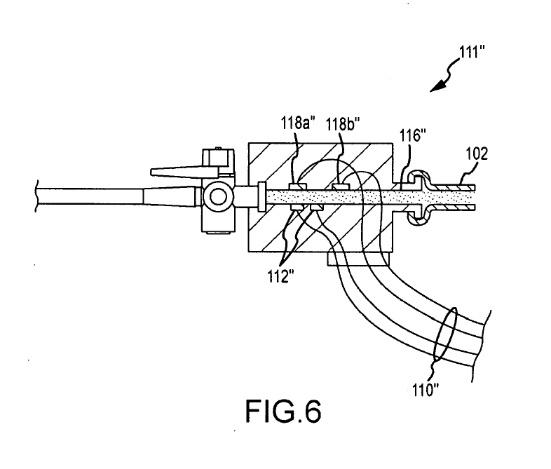

FIG. 6 is a side cross-sectional view of another sensor/detector 111″ for an oximeter which may be used with the catheter handle shown in FIG. 4. In FIG. 6, previously described parts are designated with the “double-prime” indicator and are not described herein again. In FIG. 6, the sensor/detector 111″ includes a near photodetector 118a″ and a far photodetector 118b″ positioned adjacent the source(s) 112″. Specifically, one photodetector may be sufficient to detect scattering light. But by adding one more photodetector at a different distance, another set of data is provided for use in the comparison, which can improve the accuracy of the blood oxygen measurement.

It is noted that although electrical wiring 110 is shown for each of the embodiments described above for connection of the sensor/detector 111 to the oximeter 108, alternatively, a wireless connection may be implemented. In any event, the electrical signals from the sensor(s) may be processed for further viewing by the user, e.g., as output on an electrical monitoring device. Preferably, the output is generated for the user in real-time (i.e., as the catheter is being positioned).

It is noted that any suitable analog and/or digital device may be implemented for processing and/or outputting information for the user. In addition, the information may be further characterized using a suitable processing device such as, but not limited to, a desktop or laptop computer. Such processing device may be implemented to receive the electrical signal generated by the oximeter and convert it to a corresponding condition and output for the user, e.g., at a display device, an audio signal, or tactile feedback or vibrations on the handle of the catheter. In any event, circuitry for conveying output to a user in one form or another may be readily provided by those having ordinary skill in the electronics arts after becoming familiar with the teachings herein.

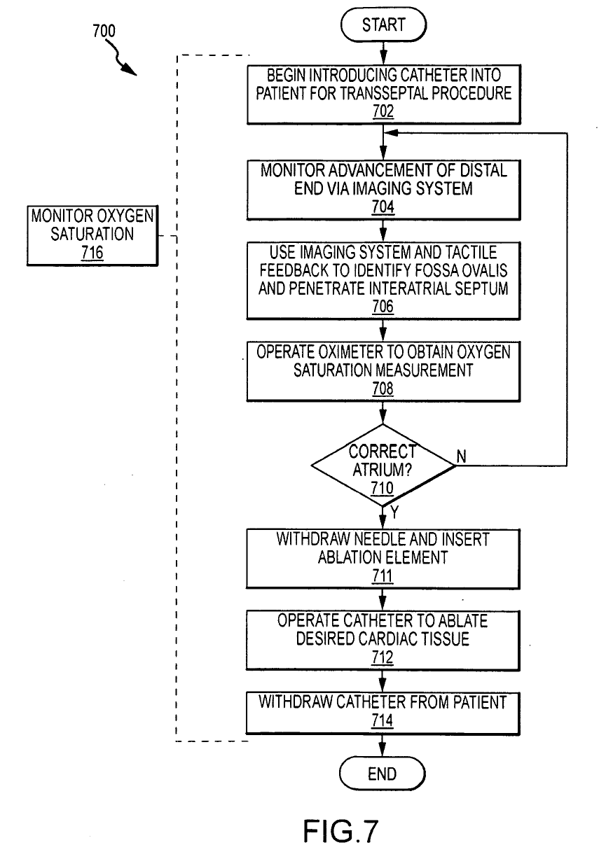

FIG. 7 is a flow chart illustrating a process 700 for performing a medical procedure in accordance with the present invention. The process 700 is initiated by beginning (702) introduction of the catheter into the patient for a transseptal procedure. For example, the catheter may be introduced into the patient's body through a vein or artery in the patient's leg, arm or neck. The catheter is then advanced through the patient's blood vessel to the patient's heart, for example, using a guide wire. Advancement of the distal end of the catheter may be monitored (704) via an imaging system. It is common to use fluoroscopic guidance and/or electrical signals together with previously acquired mapping information in this regard.

Once the distal end of the catheter has reached the patient's heart, the imaging system and tactile feedback can be used to identify (706) the fossa ovalis and to penetrate the interatrial septum. As noted above, the fossa ovalis is the thinnest portion of the interatrial septum and is generally the preferred location for penetrating the interatrial septum. Because this location is the thinnest part of the septum and generally the most compliant location, experienced physicians can identify this location via tactile feedback. The imaging system may also assist in this regard. In addition, some systems can assist in identifying the fossa ovalis based on electrical measurements of the tissue such as impedance measurements. In any event, once the physician is confident that the fossa ovalis has been identified, the distal end of the catheter is advanced to penetrate the interatrial septum.

As discussed above, successful penetration of the interatrial septum will be positively indicated by the oxygen saturation measurements from the oximeter at the proximal end of the catheter. In this regard, the physician may view the current oxygen saturation measurements after penetration of the interatrial septum to verify proper positioning of the distal end of the catheter. Alternatively, the physician may monitor the pulse oximeter readings during penetration of the interatrial septum to identify a change in oxygen saturation confirming penetration of the interatrial septum. As a still further alternative, as discussed above, such monitoring of oxygen saturation may be automated such that an indication can be provided to the physician upon penetration of the interatrial septum.

In this manner, the physician determines (710) whether the distal end of the catheter is in the correct atrium. If the distal end of the catheter is in the correct atrium, the physician can then withdraw the needle and insert the ablation element (711); and operate (712) the catheter to ablate the desired cardiac tissue or otherwise perform a desired medical procedure. Otherwise, the physician continues to manipulate the catheter to attempt to attain the proper positioning. When the procedure is complete, the physician withdraws (714) the catheter from the patient. As shown, the oximetry measurements are not limited to positioning the distal end of the catheter in the correct atrium but may be monitored (716) throughout the procedure. For example, the oxygen saturation measurements may be monitored to provide an indication of patient health thereby eliminating the need for an external pulse oximeter. In addition, the oxygen saturation measurements may be monitored throughout the procedure as a further indication that the catheter is at the expected location, e.g., within a vein, artery or the like.

Although embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A catheter system for use in positioning a distal end of a catheter body at a desired location in a patient's body, comprising:
a needle provided at the distal end of the catheter body to withdraw blood from the patient's body, the needle fluidically connected to a proximal end of the catheter body;
a measurement device provided at the proximal end of the catheter body, the measurement device configured to receive blood withdrawn by the needle for measuring a blood gas value of the blood for use in positioning the distal end of the catheter body at the desired location in the patient's body.

2. The system of claim 1, wherein the measurement device includes at least one detector configured for measuring the blood gas value.

3. The system of claim 1, wherein the measurement device includes at least one sensor configured for measuring the blood gas value.

4. The system of claim 3, wherein the sensor includes at least one LED for generating optical signals for measuring the blood gas value.

5. The system of claim 3, wherein the sensor includes at least one infrared (IR) device for generating IR signals for measuring the blood gas value.

6. The system of claim 1, wherein the measurement device converts optical signals to electrical signals representative of the blood gas value.

7. The system of claim 1, further comprising a processor configured to determine the blood gas value based on output from the measurement device.

8. The system of claim 7, wherein the processor correlates the blood gas value to a position of the catheter in the patient's body.

9. The system of claim 7, wherein the processor correlates a threshold change in the blood gas value to a position of the catheter in the patient's body.

10. The system of claim 1, wherein the measurement device is operable with a conventional catheter.

11. The system of claim 10, wherein the needle for withdrawing the blood from the patient's body is the same needle used for piercing a heart wall in the patient's body during positioning of the catheter body in the patient's body.

12. A catheter system comprising:
a catheter body having a proximal end and a distal end, the proximal end having a handle for positioning the distal end in a patient's body,
a needle insertable through the catheter body to the distal end of the catheter body, the needle configured to withdraw blood from the patient's body near the distal end of the catheter body;
a measurement device provided at the proximal end of the catheter body, the measurement device measuring a blood gas value of the blood; and
an output device operatively associated with the measurement device, the output device configured to output information based on the blood gas value for use in positioning the distal end of the catheter body at the desired location in the patient's body.

13. The system of claim 12, wherein the measurement device includes at least one detector and at least one sensor configured for measuring the blood gas value.

14. The system of claim 12, wherein the measurement device utilizes at least one of optical and infrared (IR) signals representative of the blood gas value.

15. The system of claim 12, wherein the measurement device is an oximeter.

16. The system of claim 12, wherein the output device is configured to determine the blood gas value based on output from the measurement device.

17. The system of claim 12, wherein the output device is configured to correlate the blood gas value to a position of the catheter in the patient's body.

18. The system of claim 12, wherein the output device is configured to detect a threshold change in the blood gas value.

19. A method for use in positioning a catheter at a desired location in a patient's body, comprising:
inserting the catheter into the patient's body;
withdrawing blood from the patient's body at a distal end of the catheter, and transferring the withdrawn blood through the catheter to a proximal end of the catheter;
measuring at least one parameter in the withdrawn blood at the proximal end of the catheter while the catheter remains inserted in the patient's body; and
using the at least one measured parameter to assist in positioning the catheter at the desired location in the patient's body.

20. The method of claim 19, wherein the at least one parameter includes at least one of an oxygen saturation value, a carbon dioxide value, and a pH value.

21. The method of claim 19, wherein measuring the at least one parameter includes at least one of an optical measurement and a chemical measurement.

22. The method of claim 19, further comprising correlating the at least one parameter to a position of the catheter in the patient's body.

23. The method of claim 19, further comprising correlating a threshold change in the at least one parameter to a position of the catheter in the patient's body.

24. The method of claim 23, wherein withdrawing the blood from the patient's body is in real-time during positioning of the catheter in the patient's body.

25. The method of claim 23, wherein withdrawing the blood from the patient's body is without having to insert a separate device within the catheter during positioning of the catheter in the patient's body.

26. The method of claim 23, wherein withdrawing the blood from the patient's body is with a needle of the catheter, the needle used for piercing a heart wall in the patient's body.

27. The method of claim 23, wherein measuring the at least one parameter is without one or more sensors and detectors of an oximeter contacting the blood.
